(19) United States
(12) Patent Application Publication
(10) Pub. No.: US 2016/0255422 A1
(43) Pub. Date: Sep. 1, 2016

(54) ELECTRONIC DEVICE AND METHOD

(71) Applicant: Kabushiki Kaisha Toshiba, Tokyo (JP)

(72) Inventors: Takaya Matsuno, Kunitachi Tokyo (JP); Takashi Sudo, Fuchu Tokyo (JP)

(21) Appl. No.: 14/957,909

(22) Filed: Dec. 3, 2015

Related U.S. Application Data
(60) Provisional application No. 62/121,019, filed on Feb. 26, 2015.

(51) Int. Cl. H04Q 9/00; G06F 1/32; A61B 5/00

(52) U.S. Cl. CPC H04Q 9/00; A61B 5/4809; G06F 1/3206

(57) ABSTRACT

According to one embodiment, a wearable electronic device includes a first sensor, a second sensor and a hardware processor. The first sensor is configured to detect a motion of a user wearing the electronic device.

The second sensor is configured to acquire biological information related to the user. The hardware processor is configured to determine whether the user is in an awake state or in an asleep state based on the detection value of the first sensor, and to control operation of at least one of the first sensor and the second sensor.

### CROSS-REFERENCE TO RELATED APPLICATIONS

This application claims the benefit of U.S. Provisional Application No. 62/121,019, filed Feb. 26, 2015, the entire contents of which are incorporated herein by reference.

### FIELD

Embodiments described herein relate generally to an electronic device and a method.

### BACKGROUND

In recent years, portable, battery-powered electronic devices such as tablets and smartphones have become widespread. Moreover, electronic devices worn on the person like wristwatches and glasses are also becoming popular. Many such wearable devices comprise a function of acquiring biological information of the wearer.

With a wearable device comprising a function of acquiring a user's biological information, there is a possibility that the information which the wearable device should acquire as biological information is different depending on whether the user wearing the device is in an awake state or an asleep state. A wearable device of this kind requires a mode change which causes the wearable device to change between a mode in which the wearable device operates to acquire biological information while the user in an awake state and a mode in which the wearable device operates to acquire biological information while the user is in an asleep state. Therefore, before shifting from an awake state to an asleep state, and after shifting from an asleep state to an awake state, the user has to perform an operation for a mode change without fail. Moreover, if the user forgets to change the mode, the wearable device will operate in the mode which does not agree with the user's condition. Therefore, there is a possibility that the battery may be wastefully consumed. Moreover, the cost will increase if a sensor, for example, is added in order to determine whether the user is in an awake state or in an asleep state.

### BRIEF DESCRIPTION OF THE DRAWINGS

A general architecture that implements the various features of the embodiments will now be described with reference to the drawings. The drawings and the associated descriptions are provided to illustrate the embodiments and not to limit the scope of the invention.

FIG. 1 is an exemplary view illustrating the external appearance of an electronic device of an embodiment.

FIG. 2 is an exemplary view illustrating the system configuration of the electronic device of the embodiment.

FIG. 3 is an exemplary flowchart illustrating the procedure for changing operation modes of the electronic device of the embodiment.

### DETAILED DESCRIPTION

Various embodiments will be described hereinafter with reference to the accompanying drawings.

In general, according to one embodiment, a wearable electronic device includes a first sensor, a second sensor and a hardware processor. The first sensor is configured to detect a motion of a user wearing the electronic device. The second sensor is configured to acquire biological information related to the user. The hardware processor is configured to determine whether the user is in an awake state or in an asleep state based on the detection value of the first sensor, and to control operation of at least one of the first sensor and the second sensor.

The electronic device of the present embodiment is realized as what is called a wearable device of a type that is put on a human body. Here, it is assumed that the electronic device is realized as a wristwatch type wearable device and is put on the joint between a forearm and a hand of the user (a wrist of the user).

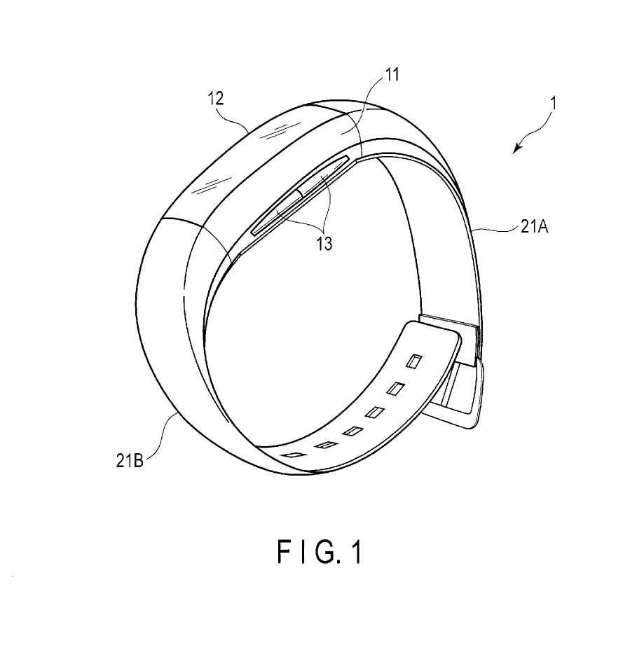

FIG. 1 is an exemplary view illustrating the external appearance of the electronic device of the embodiment. The wearable device 1 comprises a main body 11. Various electronic parts are incorporated in the main body 11. A display 12 like a liquid crystal display (LCD) is arranged at the upper surface of the main body 11. The display 12 may be a touch screen display which can detect a contact of a finger or the like to the display screen surface. Moreover, manual operation buttons 13 are arranged on one side of the main body 11.

The wearable device 1 further comprises belts (bands) 21A and 21B for putting the main body 11 on a part of a human body (a wrist). Each of the belts 21A and 21B is made of a flexible member.

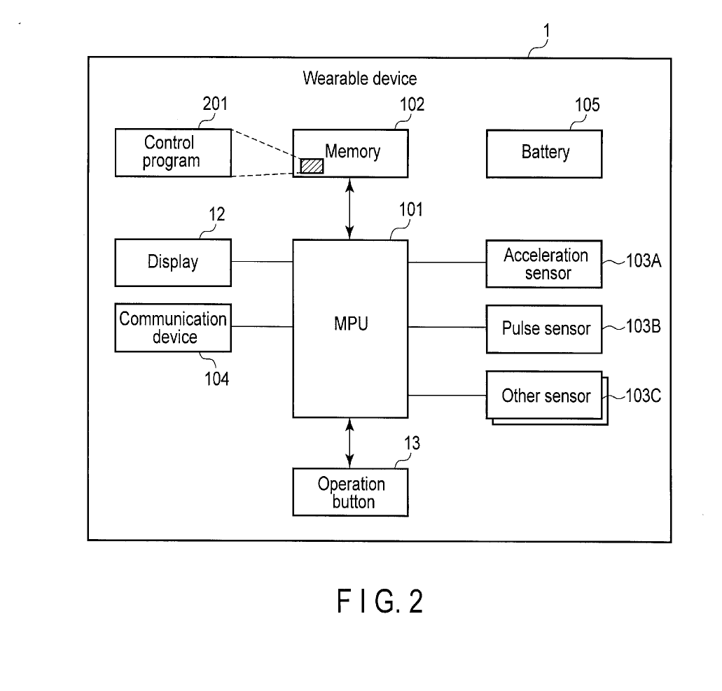

FIG. 2 is an exemplary view illustrating the system configuration of the wearable device 1.

In addition to the display 12 and the manual operation buttons 13 illustrated in FIG. 1, as illustrated in FIG. 2, a micro processing unit (MPU) 101, a memory 102, an acceleration sensor 103A, a pulse sensor 103B, further sensors 103C, a communication device 104, a battery 105, etc., are arranged at the main body 11 of the wearable device 1. It should be noted that the battery 105 is removably housed in the main body 11 and that the various components of the wearable device 1 operate with the electric power supplied from the battery 105.

MPU 101 is a processor which performs data processing using the data acquired by various sensors (103A, 103B, 103C) in accordance with the description of a control program 201 stored in the memory 102. Moreover, the MPU 101 performs various kinds of processing, including user interface processing for the display 12 and the manual operation buttons 13, and communication processing for allowing the communication device 104 to communicate with the external device. The user interface processing includes the processing which turns on and off the electric power source of the wearable device 1 according to the operation of the manual operation buttons 13. The communication device 104 is a wireless communication module which transmits and receives data in accordance with the procedure which conforms to IEEE 802.11 standard, for example.

The MPU 101 can calculate the number of steps taken by the user wearing the wearable device 1 using the data acquired by the acceleration sensor 103A. Moreover, upon counting the number of steps, the MPU 101 can determine whether the user is in a walk state or in a run state using the data acquired by the acceleration sensor 103A. It can also compute the moving distance and the calorie consumption respectively covered by and caused by the walk or run. The data processing using the data acquired by the acceleration sensor 103A makes it possible for the MPU 101 to determine various further conditions of the user wearing the wearable device 1.

Moreover, the data acquired by the pulse sensor 103B makes it possible for the MPU 101 to determine the depth of the sleep while the user wearing the wearable device 1 is sleeping (whether the user is in a REM sleep state or in a non REM sleep state) and to supervise a sleep cycle (rhythm) of the user. The data related to the human body, such as the pulse, is called biological information, for example.

Furthermore, the MPU 101 can determine various conditions of the user wearing the wearable device 1 by use of the data acquired by a plurality of the other sensors 103C. As the plurality of the other sensors 103C, a gyroscope sensor, a GPS sensor, an atmospheric pressure sensor, etc., can be used. These sensors make it possible to much more accurately determine the action of the user wearing the wearable device 1.

As mentioned above, the various components of the wearable device 1 operate with the electric power from the battery 105. Since the electric power of the battery 105 is limited, it is required that the various components should be operated efficiently and that the power consumption of the various components should be reduced. Let us reconsider from this viewpoint the data processing of the MPU 101 using the data acquired by the various sensors (103A, 103B, and 103C). For example, the data processing using the data acquired by the acceleration sensor 103A should be mainly performed while the user who wears the wearable device 1 is in an awake state. In contrast, the data processing using the data acquired by the pulse sensor 103B, for example, should be mainly performed while the user wearing the wearable device 1 is in an asleep state. Moreover, it may safely be said that the data processing using the data acquired by the plurality of the other sensors 103C should be mainly performed while the user wearing the wearable device 1 is in an awake state. Therefore, it is desirable to switch the operational modes of the wearable device 1 between the case where the user is in an awake state and the case where the user is in an asleep state.

However, if the user is required to switch the operational modes of the wearable device 1 every time the user shifts his or her states from an awake state to an asleep state or from an asleep state to an awake state, user friendliness will become worse. Moreover, if the user forgets to change the operational modes, the data processing which should be performed while the user is in an asleep state will be performed while the user is in an awake state, for example. Conversely, the data processing which should be performed while the user is in an awake state will be performed while the user is in an asleep state. Accordingly, the electric power of the battery 105 will be wastefully consumed. Furthermore, the addition of a further sensor, etc., in order to determine whether the user is in an awake state or in an asleep state will cause a cost hike. Therefore, the wearable device 1 of the present embodiment is configured to adaptively switch its operational modes despite with the existing structure.

MPU 101 computes body movement amount A[f] using the data acquired by the acceleration sensor 103A. Body movement amount A[f] is acquired by monitoring the data acquired by the acceleration sensor 103A, and is the number of times in which the acceleration greater than or equal to a predetermined threshold (for example, 0.01 G) occurs within a previously determined epoch time. Moreover, the MPU 101 computes the feature amount based on both computed body movement amount A[f] and determination value S[f] of a Cole algorithm. A Cole equation is illustrated below.

$$S [ f ] = 0.00001 ( 404 \times A [ f - 4 ] + 598 \times A [ f - 3 ] + 326 \times A [ f - 2 ] + 441 \times A [ f - 1 ] + 1408 \times A [ f ] + 508 \times A [ f + 1 ] + 350 \times A [ f + 2 ] ) .$$

In the Cole algorithm, S[f]≧1 indicates awake and S[f]<1 indicates sleep.

In the feature amount calculation, the feature amount is calculated based on S[f] and A[f], the latter being calculated in the previously determined epoch unit. The feature amount includes differences between A[f] and A[f−n], and the average, variance, standard deviation, correlation coefficient, etc., of the differences, for example. “n” is a natural number, and is determined by the capacity of the memory 102 and the permissible delay time required for calculation, for example. The MPU 101 determines whether the user is in an awake state or in an asleep state based on the comparison with each of the feature amount previously acquired while the user was in an awake state and the feature amount previously acquired while the user was in an asleep state. It should be noted that what is illustrated here as an example uses a Cole equation, but another expression such as an AW2 expression, for example, may be used. An AW2 expression is illustrated below.

$$S [ f ] = 0.0033 ( 1.06 \times A [ f - 4 ] + 0.54 \times a [ f - 3 ] + 0.58 \times A [ f - 2 ] + 0.76 \times A [ f - 1 ] + 2.3 \times A [ f ] + 0.74 \times A [ f + 1 ] + 0.67 \times A [ f + 2 ] )$$

MPU 101 automatically switches the operational modes of the wearable device 1 whenever the shift from an awake state to an asleep state or the shift from an asleep state to an awake state is detected based on the determined result. It should be noted that the method for determining whether the user who wears the wearable device 1 is in an awake state or in an asleep state using the data acquired by the acceleration sensor 103A is not limited to the above explained method but various methods may be employed.

When the shift from an awake state to an asleep state is detected, the MPU 101 turns on the pulse sensor 103B (and makes it in an operating state) in order to determine the depth of the sleep based on autonomic nerves analysis, for example. Moreover, the sampling rate (sampling frequency) of the acceleration sensor 103A is made low, for example. The reason for taking these actions is as follows: When the user is in an awake state, it is necessary to determine various conditions of the user making use of the data acquired by the acceleration sensor 103A. In contrast, when the user is in an asleep state, all that should be done is to analyze the asleep state of the user and to detect the shift from an asleep state to an awake state.

What is more, the detection of the shift from an asleep state to an awake state should cause the MPU 101 to turn off the pulse sensor 103B (idle state), for example, and furthermore to make the sampling rate (sampling frequency) of the acceleration sensor 103A to return (to a high state), for example.

Besides taking these actions, the MPU 101 dynamically executes ON/OFF control and sampling rate control for each of the sensors 103C whenever the shift from an awake state to an asleep state or from an asleep state to an awake state is detected.

Moreover, it is possible to configure the MPU 101 to control dynamically not only the aforementioned various sensors (103A, 103B, 103C) but the display 12 and the communication device 102 whenever the shift from an awake state to an asleep state and from an asleep state to an awake state is detected.

The wearable device 1 of the present embodiment determines whether the user is in an awake state or in an asleep state using the data acquired by the (existing) acceleration sensor 103A and automatically changes its own operational modes in an adaptive manner. Therefore, the wearable device 1 in the present embodiment achieves both the elimination of explicit mode change operation by the user without increasing cost and the promotion of reduction in power consumption according to change in situation.

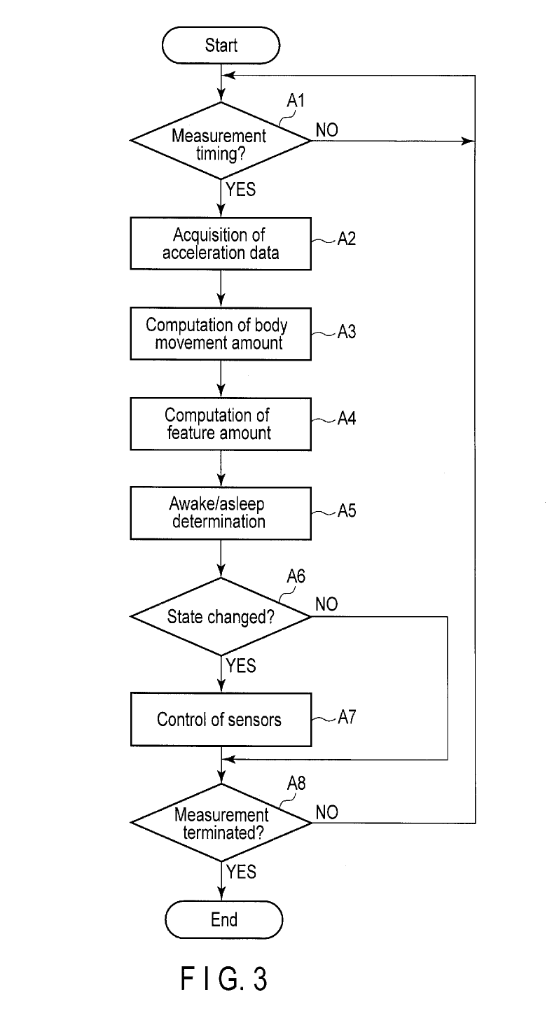

FIG. 3 is an exemplary flowchart illustrating the procedure for changing the operation modes of the wearable device 1 of the present embodiment.

At specified measurement timing (YES in block Al), the MPU 101 acquires data from the acceleration sensor 103A (block A2). The MPU 101 computes the amount of body movement of the user wearing the wearable device 1 based on the data acquired from the acceleration sensor 103A (block A3). Subsequently, the MPU 101 computes the feature amount based on the computed amount of body movement (block A4). Then, the MPU 101 determines based on the computed feature amount whether the user wearing the wearable device 1 is in an awake state or in an asleep state (block A5).

When the MPU 101 detects the shift from an awake state to an asleep state or from an asleep state to an awake state based on the determined result (YES in block A6), the MPU 101 executes change in the operational mode of the wearable device 1 including control of the various sensors (103A, 103B, 103C) (block A7). The MPU 101 repeats the process from block Al to the present block if the user does not give any explicit instructions to terminate the measurement (NO in block A8). When explicit instructions to terminate the measurement are given by the user (YES in block A8), the MPU 101 stops the operation of the wearable device 1.

As mentioned above, the present embodiment makes it possible that the wearable device 1 will adaptively change its operational modes despite the existing structure. That is, the wearable device 1 of the present embodiment makes it possible to make unnecessary explicit mode change operation by the user without causing a cost hike and to promote reduction in power consumption according to a situation.

Various functions described herein to explain the present embodiment may be realized by processing circuits (hardware processors). A programmed processor such as a central processing unit (CPU) may be enumerated as an example of the processing circuit. The processor performs the described functions by executing programs stored in the memory. The processor may be a microprocessor containing an electric circuit. The processing circuit includes, for example, a digital signal processor (DSP), an application specific integrated circuit (ASIC), a microcontroller, a controller, and other electric circuit components.

It should be noted that the operational procedures of the present embodiment can be realized by a computer program, which makes it possible to easily accomplish the same effects as the embodiment only to install the computer program in a computer through a computer readable storage medium storing the computer program and to cause the computer to execute the installed computer program.

The various modules of the systems described herein can be implemented as software applications, hardware and/or software modules, or components on one or more computers, such as servers. While the various modules are illustrated separately, they may share some or all of the same underlying logic or code.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Claims what is claimed is:

1. A wearable electronic device comprising:
a first sensor configured to detect a motion of a user wearing the electronic device;
a second sensor configured to acquire biological information related to the user; and
a hardware processor configured to determine whether the user is in an awake state or in an asleep state based on the detection value of the first sensor, and to control operation of at least one of the first sensor and the second sensor.

2. The device of claim 1, wherein the hardware processor is configured to control a start or a stop of the second sensor according to whether a state of the user changes from the awake state to the asleep state or from the asleep state to the awake state.

3. The device of claim 1, wherein the hardware processor is configured to control a sampling rate of at least one of the first sensor and the second sensor according to whether a state of the user changes from the awake state to the asleep state or from the asleep state to the awake state.

4. The device of claim 1, wherein the first sensor comprises an acceleration sensor.

5. The device of claim 1, wherein the second sensor comprises a pulse sensor.

6. The device of claim 1, wherein the first sensor and the second sensor operate with electric power from a battery.

7. A method for a wearable electronic device, the method comprising:
detecting a motion of a user wearing an electronic device by a first sensor;
acquiring biological information related to the user by a second sensor;
determining whether the user is in an awake state or in an asleep state based on a detection value of the first sensor; and
controlling operation of at least one of the first sensor and the second sensor based on the determined result.

8. The method of claim 7, wherein the controlling comprises controlling a start or a stop of the second sensor according to whether a state of the user changes from the awake state to the asleep state or from the asleep state to the awake state.

9. The method of claim 7, wherein the controlling comprises controlling a sampling rate of at least one of the first sensor and the second sensor according to whether a state of the user changes from the awake state to the asleep state or from the asleep state to the awake state.

10. The method of claim 7, wherein the first sensor comprises an acceleration sensor.

11. The method of claim 7, wherein the second sensor comprises a pulse sensor.

12. The method of claim 7, wherein the first sensor and the second sensor operate with electric power from a battery.
